SEQUENCE LISTING (7 sequences)

SEQ ID NO: 1 - protein, 182 aa, Homo sapiens
GSHSMRYFSAAVSRPGRGEPRFIAMGYVDDTQFVRFDSDSACPRMEPRAPWVEQEGPEYWEEETRNTKAHAQTDRMNLQTLRGYYNQSEANPPKTHVTHHPVFDYEATLRCWALGFYPAEIILTWQRDGEDQTQDVELVETRPAGDGTFQKWAAVVVPSGEEQRYTCHVQHEGLPEPLMLRW

SEQ ID NO: 2 - DNA, 546 bp, Artificial
  Base sequence having base sequence 1-18 of SEQ ID NO5 and base sequence 19-546 of SEQ ID NO2
ggatcacattcaatgagatatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccaacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatgg

SEQ ID NO: 3 - DNA, 41 bp, Artificial
  5′-primer
atgggtagtcatagtatgcgttattttagcgcggccgtgag

SEQ ID NO: 4 - DNA, 41 bp, Artificial
  3′-primer
ctcacggccgcgctaaaataacgcatactatgactacccat

SEQ ID NO: 5 - protein, 700 aa, Mus musculus
MSCTFTALLRLGLTLSLWIPVLTGSLPKPILRVQPDSVVSRRTKVTFLCEETIGANEYRLYKDGKLYKTVTKNKQKPENKAEFSFSNVDLSNAGQYRCSYSTQYKSSGYSDLLELVVTGHYWTPSLLAQASPVVTSGGYVTLQCESWHNDHKFILTVEGPQKLSWTQDSQYNYSTRKYHALFSVGPVTPNQRWICRCYSYDRNRPYVWSPPSESVELLVSGNLQKPTIKAEPGSVITSKRAMTIWCQGNLDAEVYFLHNEKSQKTQSTQTLQEPGNKGKFFIPSVTLQHAGQYRCYCYGSAGWSQPSDTLELVVTGIYEYYEPRLSVLPSPVVTAGGNMTLHCASDFPYDKFILTKEDKKFGNSLDTEHISSSGQYRALFIIGPTTPTHTGAFRCYGYYKNAPQLWSVPSALQQILISGLSKKPSLLTHQGHILDPGMTLTLQCFSDINYDRFALHKVGGADIMQHSSQQTDTGFSVANFTLGYVSSSTGGQYRCYGAHNLSSEWSASSEPLDILITGQLPLTPSLSVQPNHTVHSGETVSLLCWSMDSVDTFILSKEGSAQQPLRLKSKSHDQQSQAEFSMSAVTSHLSGTYRCYGAQDSSFYLLSSASAPVELTVSGPIETSTPPPTMSMPLGGLHMYLKALIGVSVAFILFLFIFIFILLRRRHRGKFRKDVQKEKDLQLSSGAEEPITRKGELQKR

SEQ ID NO: 6 - DNA, 2100 bp, Mus musculus
atgtcctgcaccttcacagccctgctccgtcttggactgactctgagcctctggatcccagtgctgacagggtccctccctaagcctatcctcagagtacagccagactctgtggtctccaggaggactaaggtgaccttcttgtgtgaagagacaattggagccaatgagtaccgcctctataaagatggaaagctatataaaaccgtaacaaagaacaaacagaagccagaaaacaaggctgaattctcattctcaaatgtagacctgagtaatgcaggtcaatatcgatgttcctacagcacccagtataaatcatcaggctacagtgacctcctggagctggtggtgacaggacactactggacacccagccttttagcccaagccagccctgtggtaacttcaggagggtatgtcaccctccagtgtgagtcctggcacaacgatcacaagttcattctgactgtagaaggaccacagaagctctcgtggacacaagactcacagtataattactctacaaggaagtaccacgccctgttctctgtgggccctgtgacccccaaccagagatggatatgcagatgttacagttatgacaggaacagaccatatgtgtggtcacctccaagtgaatccgtggagctcctggtctcaggtaatctccaaaaaccaaccatcaaggctgaaccaggatctgtgatcacctccaaaagagcaatgaccatctggtgtcaggggaacctggatgcagaagtatattttctgcataatgagaaaagccaaaaaacacagagcacacagaccctacaggagcctgggaacaagggcaagttcttcatcccttctgtgacactacaacatgcagggcaatatcgctgttattgttacggctcagctggttggtcacagcccagtgacaccctggagctggtggtgacaggaatctatgaatactatgaacccaggctgtcagtactgcccagccctgtggtgacagctggagggaacatgacactccactgtgcctcagactttccctacgataaattcattctcaccaaggaagataagaaattcggcaactcactggacacagagcatatatcttctagtggacagtaccgagccctgtttattataggacccacaaccccaacccatacaggggcattcagatgttacggttactacaagaatgccccacagctgtggtcagtacctagtgctctccaacaaatactcatctcagggctgtccaagaagccctctctgctgactcaccaaggccatatcctggaccctggaatgaccctcaccctgcagtgtttctctgacatcaactatgacagatttgctctgcacaaggtggggggagctgacatcatgcagcactctagccagcagactgacactggcttctctgtggccaacttcacactgggctatgtgagtagctccactggaggccaatacagatgctatggtgcacacaacctctcctctgagtggtcagcctccagtgagcccctggacatcctgatcacaggacagctccctctcactccttccctctcagtgcagcctaaccacacagtgcactcaggagagaccgtgagcctgctgtgttggtcaatggactctgtggatactttcattctgtccaaggagggatcagcccagcaacccctgcgactaaaatcaaagtcccatgatcagcagtcccaggcagaattctccatgagtgctgtgacctcccatctctcaggcacctacaggtgctatggagctcaagactcatctttctacctcttgtcatctgccagtgcccctgtggagctcacagtctcaggacccatcgaaacctctaccccgccacccacaatgtccatgccactaggtggactgcatatgtacctgaaggctctcattggagtgtctgtggccttcatcctgttcctcttcatcttcatcttcattcttcttcgacgaagacatcggggaaaattcaggaaagatgtccagaaagagaaagacttgcaactttcttcaggagctgaagagcccataaccaggaaaggagaactccagaagagg

SEQ ID NO: 7 - protein, 10 aa, ARTIFICIAL
  Mart1 peptide
ELAGIGILTV

(19) United States
(12) Patent Application Publication
(10) Pub. No.: US 2021/0115137 A1
(43) Pub. Date: Apr. 22, 2021

(54) PROPHYLACTIC OR THERAPEUTIC AGENT FOR TUMORS, PD-L1 INHIBITOR, SCREENING METHOD FOR PROPHYLACTIC OR THERAPEUTIC AGENT FOR TUMORS, AND SCREENING METHOD FOR PD-L1 INHIBITOR

(71) Applicant: National University Corporation Hokkaido University, Hokkaido (JP)

(72) Inventors: Katsumi Maenaka, Hokkaido (JP); Kimiko Kuroki, Hokkaido (JP); Ami Takahashi, Hokkaido (JP)

(21) Appl. No.: 16/980,090

(22) PCT Filed: Mar. 13, 2019
(86) PCT No.: PCT/JP2019/010364

§ 371 Date: Dec. 15, 2020

(30) Foreign Application Priority Data
Mar. 13, 2018 (JP) 2018-046024

(51) Int. Cl. C07K 16/28; G01N 33/574

(52) U.S. Cl. CPC C07K 16/2803; G01N 2500/02; C07K 2317/76; G01N 33/574

(57) ABSTRACT

Provided is prophylactic or therapeutic agents for tumors containing an inhibitor of the interaction between HLA-G2 and leukocyte Ig-like receptor B2 (LILRB2) as an active ingredient.

### TECHNICAL FIELD

The present disclosure relates to prophylactic or therapeutic agents for tumors, PD-L1 inhibitors, methods of screening for prophylactic or therapeutic agents for tumors, and methods of screening for PD-L1 inhibitors.

### BACKGROUND ART

In recent years, the development of anticancer drugs that target immune checkpoint signaling pathways including Programmed Cell Death 1 (PD-1)/Programmed Cell Death 1 Ligand-1 (PD-L1, also known as Programmed death-ligand 1) signaling has been actively progressed. Although cancers cause suppressed immune system in vivo, antibody therapeutics that recovers the original activity of such an immune system has come to play an important role in cancer treatment.

In addition, search for novel target molecules and development of novel drugs have been actively progressed mainly from CD28/B7 family members including PD-1/PD-L1. PD-L1 is a cell surface molecule that is originally expressed on antigen-presenting cells and engaged in the control of T-cell activation. However, PD-L1, particularly expressed on tumor cells in cancers with poor prognosis, inhibits activation of T-cell for the cancer cells, inducing suppression of tumor immunity (Non Patent Literature 1).

Nivolumab (product name: OPDIVO) and pembrolizumab (product name: KEYTRUDA) are commercially available as anti-PD-1 antibody drugs and reported to exhibit favorable effects in certain tumors (Non Patent Literature 2).

### CITATION LIST

### Non Patent Literature

Non Patent Literature 1: Hamanishi J1, Mandai M, Iwasaki M, Okazaki T, Tanaka Y, Yamaguchi K, Higuchi T, Yagi H, Takakura K, Minato N, Honjo T, Fujii S. Programmed cell death 1 ligand 1 and tumor-infiltrating CD8+ T lymphocytes are prognostic factors of human ovarian cancer. Proc Natl Acad Sci USA. 2007 Feb. 27; 104(9): 3360-5. Epub 2007 Feb. 21.
Non Patent Literature 2: Wang Y1, Wu L1, Tian C2, Zhang Y3. PD-1-PD-L1 immune-checkpoint blockade in malignant lymphomas. Ann Hematol. 2018 February; 97 (2): 229-237. doi: 10.1007/s00277-017-3176-6. Epub 2017 Nov. 11.

### SUMMARY OF INVENTION

### Technical Problem

However, anti-PD-1 antibody drugs that directly inhibit PD-L1 still have had problems with side effects regarding autoimmunity and reduction in efficacy due to anti-antibody production.

In view of the above circumstances, an objective of the present disclosure is to provide novel prophylactic or therapeutic agents for tumors focusing on inhibition of the interaction between human leukocyte antigen (HLA)-G2 (HLA-G2) and leukocyte Ig-like receptor B2 (LILRB2), PD-L1 inhibitors, and methods of screening therefor.

### Solution to Problem

To achieve the objective described above, a prophylactic or therapeutic agent for tumors according to the first aspect of the present disclosure contains an inhibitor of the interaction between HLA-G2 and leukocyte Ig-like receptor B2 (LILRB2) as an active ingredient.

For example, the interaction inhibitor is an anti-LILRB2 antibody.

A programmed cell death ligand 1 (PD-L1) inhibitor according to the second aspect of the present disclosure contains an inhibitor of the interaction between HLA-G2 and leukocyte Ig-like receptor B2 (LILRB2) as an active ingredient.

For example, the interaction inhibitor is an anti-LILRB2 antibody.

A method of screening for prophylactic or therapeutic agents for tumors according to the third aspect of the present disclosure includes the steps of:

determining a degree of binding between HLA-G2 and leukocyte Ig-like receptor B2 (LILRB2) in the presence and absence of a test substance;

comparing the degree in the presence of the test substance with the degree in the absence of the test substance; and

identifying the test substance as a prophylactic or therapeutic agent for tumors when the degree in the presence of the test substance is lower than the degree in the absence of the test substance.

A method of screening for programmed cell death ligand 1 (PD-L1) inhibitors according to the fourth aspect of the present disclosure includes:

determining a degree of binding between HLA-G2 and leukocyte Ig-like receptor B2 (LILRB2) in the presence and absence of a test substance;

comparing the degree in the presence of the test substance with the degree in the absence of the test substance; and

identifying the test substance as a prophylactic or therapeutic agent for tumors when the degree in the presence of the test substance is lower than the degree in the absence of the test substance.

### Advantageous Effects of Invention

According to the present disclosure, novel prophylactic or therapeutic agents for tumors focusing on inhibition of the interaction between HLA-G2 and LILRB2, PD-L1 inhibitors, and methods of screening therefor can be provided.

### BRIEF DESCRIPTION OF DRAWINGS

FIG. 1A shows results of gel filtration chromatography of HLA-G2; and FIG. 1B shows results of SDS-PAGE of a fraction taken from the peak (arrow portion) in FIG. 1A;

FIG. 2A shows results of gel filtration chromatography of a PIR-B preparation; and FIG. 2B shows results of SDS-PAGE of a fraction taken from the peak (arrow portion) in FIG. 2A followed by Western blotting using an anti-FLAG antibody for detection;

FIG. 3 shows surface plasmon resonance (SPR) analysis of HLA-G2 to PIR-B;

FIG. 4 shows a process for preparing monocytes from human PBMCs; FIG. 4A shows preparation of monocytes; FIG. 4B shows flow cytometry for selection of a living monocyte population; FIG. 4C shows confirmation of expression of LILRB2 on selected monocytes;

FIG. 5 shows results of flow cytometry analysis of cell surface molecules on monocytes expressing human LILRB2 after incubation with HLA-G2 for 2 days;

FIG. 6 shows results of ELISA analysis of cytokines in monocytes expressing human LILRB2 after incubation with HLA-G2 for 2 days; FIG. 6A shows IL-6 production; FIG. 6B shows IL-10 production;

FIG. 7 shows results of Western blotting analysis of signal activation in monocytes expressing human LILRB2 after incubation with HLA-G2 for 2 days;

FIG. 8 shows results of evaluation of an antibody that blocks the interaction between LILRB2 and HLA-G2; FIG. 8A shows response over time through three additions of 27D6 antibody; FIG. 8B shows comparison of responses to an injection of HLA-G2 with LILRB2 alone and with LILRB2 sufficiently bound by 27D6 antibody (LILRB2+27D6);

FIG. 9 shows effects of blocking with 27D6 antibody on functional changes in monocytes expressing human LILRB2 after incubation with HLA-G2 for 2 days; FIG. 9A shows Western blotting analysis of signal activation, FIG. 9B shows ELISA analysis of IL-6 production; and FIG. 9C shows ELISA analysis of IL-10 production;

FIG. 10 shows results of flow cytometry analysis of cell surface molecules on IL-4-DCs after incubation with HLA-G2 for 6 days;

FIG. 11 shows results of ELISA analysis of cytokines in IL-4-DCs after incubation with HLA-G2 for 3 days; FIG. 11A shows IL-6 production; FIG. 11B shows IL-10 production;

FIG. 12 shows results of flow cytometry analysis of cell surface molecules on IFN-DCs after incubation with HLA-G2 for 2 days;

FIG. 13 shows results of ELISA analysis of cytokines in IFN-DCs after incubation with HLA-G2 for 2 days; FIG. 13A shows IL-6 production; FIG. 13B shows IL-10 production;

FIG. 14A shows an outline of autologous mixed lymphocyte reaction experiment in IFN-DCs using CD8⁺ T-cells; and FIG. 14B shows results thereof,

FIG. 15 is schematic illustration of LILRB2-HLA-G2 binding inhibition experiments for increasing PD-L1 expression;

FIG. 16A shows results of LILRB2-HLA-G2 binding inhibition experiments for increasing PD-L1 expression; FIG. 16B shows a graph comparing MFIs; and FIG. 16C shows a graph comparing the decreases in PD-L1-positive cells; and

FIG. 17 shows a tumor immunity mechanism induced by blocking the interaction between HLA-G2 and LILRB2.

### DESCRIPTION OF EMBODIMENTS

First, prophylactic or therapeutic agents for tumors in the present embodiments will be described in detail.

In the present embodiments, the prophylactic or therapeutic agent for tumors comprises an inhibitor of an interaction between HLA-G2 and leukocyte Ig-like receptor B2 (LILRB2) as an active ingredient.

In the present description, inhibitors of the interaction between HLA-G2 and LILRB2 may be referred to as “HLA-G2-LILRB2 interaction inhibitors.”

The present inventors studied functions of signaling between HLA-G2 and LILRB2, and found that stimulation with HLA-G2 in monocytes derived from human peripheral blood led to decrease in the expression of CD86 and HLA-DR, as well as clear increase in the expression of PD-L1. Although down-regulation of CD86 and HLA-DR by stimulation with HLA-G1 isoform has been reported, study with HLA-G2 is the first time. Also, up-regulation of PD-L1 has not been reported, even in the case using HLA-G1 isoform. Thus, the present inventors have newly found that not only down-regulation of immune activation molecules but also up-regulation of PD-L1 are important for the function of HLA-G2 to induce immunosuppression. In addition, the present inventors have found that stimulation with HLA-G2 causes up-regulation of an intracellular protein Indoleamine-2,3-dioxygenase-1 (IDO) involved in inhibition of T-cell activation; up-regulation of Interleukin-10 (IL-10) that is thought to be an upstream signal of IDO; up-regulation of Interleukin-6 (IL-6) that has been reported to be involved in induction of immunosuppression in monocytes and antigen-presenting cells; and enhanced phosphorylation of Signal Transducer and Activator of Transcription 3 (STAT3). The present inventors have also found that a blocking experiment of the LILRB2-HLA-G2 interaction results in functional recovery in some of the results. From the above results, the present inventors believe that blocking the HLA-G2-LILRB2 interaction can induce tumor immunity via PD-L1, thereby completing the present disclosure. The present disclosure aims to develop small molecule and antibody drugs that inhibit up-regulation of PD-L1 via a receptor, LILRB2, on tumor cells or antigen-presenting cells without directly inhibiting the PD-1/PD-L1 interaction.

HLA-G2 is one of splicing isoforms of Human Leukocyte Antigen (HLA)-G that is a non-classical MHC class I molecule. For HLA-G2, NCBI includes the gene sequences of full-length human-derived HLA-G (=HLA-G1) as NM_002127.5. Among the gene sequences, the “amino acid region 1-90” in SEQ ID NO: 1 corresponds to the amino acid sequence of the α1 domain, and the “amino acid region 91-180” corresponds to the amino acid sequence of the α3 domain.

LILRB2 is originally found as a receptor molecule that is expressed on antigen-presenting cells and recognize human leukocyte antigen (HLA) class I molecules on self cells as ligands, thereby participating in acquisition of self-tolerance. LTLRB2 broadly recognizes classical and non-classical HLA class I molecules as ligands. Among them, it has been demonstrated that LTLRB2 strongly binds to HLA-G2 with a dissociation constant of nM order (Kuroki K, Mio K, Takahashi A, Matsubara H, Kasai Y, Manaka S, Kikkawa M, Hamada D, Sato C, Maenaka K., Cutting Edge: Class II-like Structural Features and Strong Receptor Binding of the Nonclassical HLA-G2 Isoform Homodimer. J Immunol. 2017 May 1; 198(9): 3399-3403. doi: 10.4049/jimmunol.1601296. Epub 2017 Mar. 27). In addition, it has been reported that LILRB2 is expressed in non-small-cell lung cancer (NSCLC) and patients with expression of LILRB2 experience poorer prognosis than patient without expression of LILRB2 (P. Zhang et al., Oncotarget. 2015), and that LILRB2 and HLA-G are expressed in NSCLC and poor prognosis is observed especially in double-positive patients (Y. Zhang et al., Tumor Biol. 2016).

HLA-G2-LILRB2 interaction inhibitors refer to substances that functions to block the interaction between HLA-G2 and LILRB2. For example, an HLA-G2-LILRB2 interaction inhibitor may be determined as a substance that functions to block the interaction when reduction of the degree of the interaction in the presence of the HLA-G2-LILRB2 interaction inhibitor is, for example, 1.1 times or more, 1.5 times or more, 1.8 times or more, 2.0 times or more than reduction of the degree of the interaction in the absence of the HLA-G2-LILRB2 interaction inhibitor. The method of measuring the degree of the interaction in the presence or absence of an HLA-G2-LILRB2 interaction inhibitor may be, for example, an interaction analysis method by Surface Plasmon Resonance (SPR) using BIACORE 3000.

The HLA-G2-LILRB2 interaction inhibitor may be, for example, a small-molecule compound, antibody, or peptide that functions to block the interaction between HLA-G2 and LILRB2, or may be, for example, a protein such as a recombinant LILRB2 receptor protein or an unidentified HLA-G2-specific receptor binding protein. The HLA-G2-LILRB2 interaction inhibitor may be an anti-LILRB2 antibody that functions to block the interaction between HLA-G2 and LILRB2.

In the present embodiments, the prophylactic or therapeutic agent for tumors comprises an HLA-G2-LILRB2 interaction inhibitor as an active ingredient and blocks the interaction between HLA-G2 and LILRB2 to induce tumor immunity via down-regulation of IDO involved in inhibition of T-cell activation, down-regulation of IL-10 that is thought to be an upstream signal of IDO, down-regulation of IL-6 involved in induction of immunosuppression in monocytes and antigen-presenting cells, and down-regulation of PD-L1. In such present embodiments, the prophylactic or therapeutic agent for tumors shows an effect of preventing or treating tumors through a comprehensive action by, for example, down-regulation of PD-L1 as well as down-regulation of IL-10 and IL-6.

In the present embodiments, the prophylactic or therapeutic agent for tumors shows a prophylactic or therapeutic effect on tumors such as breast cancer, liver cancer, non-small-cell lung cancer, adrenocortical carcinoma, anal cancer, bile duct cancer, bladder cancer, cervical cancer, colorectal cancer, endometrial cancer, esophageal cancer, Ewing's tumor, gallbladder cancer, Hodgkin's disease, hypopharyngeal cancer, laryngeal cancer, oral cavity cancer, non-Hodgkin's lymphoma, melanoma, mesothelioma, multiple myeloma, ovarian cancer, pancreas cancer, prostate cancer, gastric cancer, testicular cancer, thyroid cancer, chronic myelogenous leukemia, and chronic lymphocytic leukemia (CLL).

In the present embodiments, the prophylactic or therapeutic agent for tumors may further comprise pharmaceutically acceptable carriers (e.g., fillers, binders, disintegrants, lubricants, stabilizer, preservatives, pH adjusting agents, flavoring agents, diluents, and injectable vehicles). The agent may also comprise a label, a nanocapsule, or the like that allows the agent to be specifically delivered to a target tissue. The agent may further comprise other therapeutically effective components such as a known anticancer agent that is effective in treatment of tumors (e.g., fluorouracil, tamoxifen, anastrozole, aclarubicin, doxorubicin, tegafur, cyclophosphamide, irinotecan, cytarabine, paclitaxel, docetaxel, epirubicin, carboplatin, cisplatin, thiotepa, or a pharmaceutically acceptable salt thereof). Alternatively, the agent may be administered in combination with such an anticancer agent.

In the present embodiments, the route of administration of the prophylactic or therapeutic agent for tumors may be, for example, oral or parenteral administration (e.g., intravenous, intraarterial, subcutaneous, intramuscular, intraperitoneal, or local administration). Exemplary dosage forms include injections, tablets, capsules, granules, syrup, emulsions, suppositories, suspensions, and sprays. Specifically, in the case of local administration, the therapeutic agent for cancers of the present disclosure can be administered directly to the cancer tissue by means of a syringe or the like after exposure of the affected area in a surgical procedure, or in the case of non-local administration, the agent can be administered into a tumor nutrient vessel.

In the present embodiments, the dose and frequency of administration of the prophylactic or therapeutic agent for tumors may vary and selected as appropriate according to the desired effect, the route of administration, the duration of the treatment, the age, body weight, and gender of the subject, and the like.

In the present embodiments, the prophylactic or therapeutic effect of the prophylactic or therapeutic agent for tumors on a tumor can be evaluated by, for example, measuring the tumorigenicity, mean survival time, and invasion to organs in the treated mammal.

Next, PD-L1 inhibitors in the present embodiments will be described in detail.

In the present embodiments, the PD-L1 inhibitor comprises an inhibitor of the interaction between HLA-G2 and LILRB2 as an active ingredient. For example, the interaction inhibitor is an anti-LILRB2 antibody.

PD-L1 is a cell surface molecule that is originally expressed on antigen-presenting cells and engaged in the control of T-cell activation. However, PD-L1, particularly expressed on tumor cells in cancers with poor prognosis, inhibits activation of T-cell for the cancer cells, inducing suppression of tumor immunity. In the present embodiments, the PD-L1 inhibitor comprises an inhibitor of the interaction between HLA-G2 and LILRB2 as an active ingredient and blocks the interaction between HLA-G2 and LILRB2 to induce tumor immunity via down-regulation of PD-L1 in tumor cells.

In the description of the PD-L1 inhibitor in the present embodiments, HLA-G2, LILRB2, HLA-G2-LILRB2 interaction inhibitors, target diseases, excipients, dosage forms, and the like are as described above.

Next, methods of screening for prophylactic or therapeutic agents for tumors in the present embodiments will be described in detail.

In the present embodiments, the methods of screening for prophylactic and/or therapeutic agents for tumors comprises the steps of:

(a) determining the degree of binding between HLA-G2 and LILRB2 in the presence and absence of a test substance;

(b) comparing the degree in the presence of the test substance with the degree in the absence of the test substance; and

(c) identifying the test substance as a prophylactic or therapeutic agent for tumors when the degree in the presence of the test substance is lower than the degree in the absence of the test substance.

The method of measuring the degree of binding between HLA-G2 and LILRB2 in the steps (a) and (b) above may be, for example, an interaction analysis method by Surface Plasmon Resonance (SPR) using BIACORE 3000.

The test substance used in the steps (a) to (c) above is not particularly restricted, and examples of the test substance include small-molecule compounds, antibodies, peptides, and recombinant proteins.

In the step (c) above, a test substance may be identified as a prophylactic or therapeutic agent for tumors when reduction of the degree of binding between HLA-G2 and LILRB2 in the presence of the test substance is, for example, 1.1 times or more, 1.5 times or more, 1.8 times or more, 2.0 times or more than reduction of the degree of binding in the absence of the test substance.

Next, methods of screening for PD-L1 inhibitors in the present embodiments will be described in detail.

In the present embodiments, the methods of screening for PD-L1 inhibitors comprises the steps of:

(a) determining a degree of binding between HLA-G2 and LILRB2 in the presence and absence of a test substance;

(b) comparing the degree in the presence of the test substance with the degree in the absence of the test substance; and

(c) identifying the test substance as a prophylactic or therapeutic agent for tumors when the degree in the presence of the test substance is lower than the degree in the absence of the test substance.

Details of the steps (a) to (c) above are as described above.

As described above, novel prophylactic or therapeutic agents for tumors focusing on inhibition of the interaction between HLA-G2 and LILRB2, PD-L1 inhibitors, and methods of screening therefor are provided.

### EXAMPLES

The present disclosure will now be described in detail with reference to Examples. However, the present disclosure is not limited thereto.

### Example 1

The following experiment was carried out to examine the functions of HLA-G2-LILRB2 signaling in human.

(Preparation of HLA-G2 Protein)

(1) Expression of α1-3 Complex as Inclusion Body in Escherichia coli

A pGMT7 vector was digested with restriction enzymes, NdeI and HindIII. A modified gene (SEQ ID NO: 2) encoding a complex of the α1 and α3 domains of the HLA-G molecule (α1-3 complex) was inserted into the vector using T4 DNA ligase to construct modified HLA-G[α1-3]-pGMT7 gene.

A modified HLA-G[α1-3]-pGMT7 plasmid was performed by the following method. PCR was first performed using the HLA-G[α1-3]-pGMT7 plasmid as a template with addition of PCR buffer solution (produced by Promega Corporation), deoxyNTPs mixture (produced by Toyobo Co., Ltd.), Forward primer at 5′ end (atgggtagtcatagtatgcgttattttagcgcggccgtgag: SEQ ID NO: 3) and Reverse primer at 3′ end (ctcacggccgcgctaaaataacgcatactatgactacccat: SEQ ID NO: 4) (each having a final concentration of 0.2 μM), and PfuTurbo DNA Polymerase (produced by Promega Corporation). The PCR reaction performed with 25 cycles of denaturation for 30 seconds at 95° C., annealing 1 minute at 60° C., and extension for 8 minutes at 68° C. After addition of DpnI (produced by New England Biolabs, Inc.) to the PCR product, the mixture was allowed to react at 37° C. for 1 hour before removing the template. The resulting solution was subjected to agarose gel electrophoresis to confirm the presence of the PCR products. Finally, the nucleic acid sequence was determined using a DNA sequencer, thereby obtaining a modified HLA-G[α1-3]-pGMT7 plasmid.

Next, Escherichia coli competent cells, ClearColi® BL21 (DE3) (chemical competent cells modified from ClearColi® BL21 (DE3) competent cells (Lucigen) by the present inventors) were transformed with the modified HLA-G[α1-3]-pGMT7 plasmid. The cells were cultured in 2× YT medium (0.5% sodium chloride, 1.6% triptone, 1% dry yeast extract (which are produced by Nacalai Tesque, Inc.)) supplemented with 100 mg/L of ampicillin at 37° C. At a time when the culture reached an OD600 of 0.4 to 0.6, 1 mM of IPTG was added, followed by induction of expression at 37° C. for 4 to 6 hours.

(2) Refolding of Inclusion Body from Escherichia coli

The bacterial suspension after induction of expression by addition of IPTG was centrifuged to collect the cells. The cells were resuspended with a Resuspension buffer (50 mM Tris pH8.0, 100 mM sodium chloride), homogenized by sonication, and centrifuged to obtain inclusion bodies. The inclusion bodies were washed well with a Triton wash buffer (0.5% Triton X-100, 50 mM Tris pH8.0, 100 mM sodium chloride) and a Resuspension buffer (50 mM TrispH8.0, 100 mM sodium chloride) and then solubilized with 6.0 M Guanidine solution (6.0 M guanidine, 50 mM MES pH6.5, 10 mM MEDTA). At this time, the UV absorbance at A280 of the HLA-G [α1-3] solution was measured to be about 70, assuming that the expression of HLA-G [α1-3] was about 100 mg/L. The inclusion bodies were refolded by a common dilution method using a Refolding buffer (0.1 M Tris pH8.0, 0.4 M L-arginine, 5 mM EDTA, 3.7 mM cystamine, 6.4 mM cysteamine) with stirring at 4° C. for 72 hours. Finally, the resulting product was concentrated, and purified by gel filtration chromatography under the following conditions.

### <Conditions of Gel Filtration Chromatography>

Column: HiLoad 26/60, Superdex 75 (60 cm, id 26 mm)

Mobile phase: 20 mM Tris-HCl, 100 mM NaCl buffer (pH8)

Flow rate: 2.5 ml/min

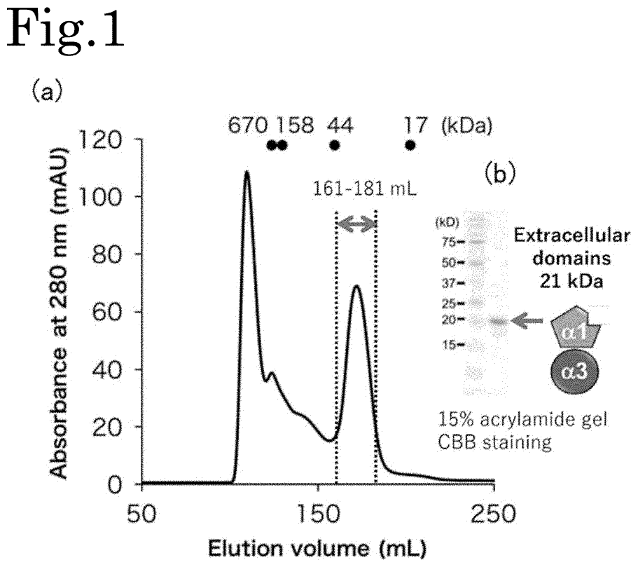

The chromatogram obtained from the gel filtration chromatography is shown in FIG. 1A. The fraction between 161-181 mL shown in FIG. 1A was taken and subjected to SDS-PAGE under non-reducing conditions (15% acrylamide gel). The results are shown in FIG. 1B. Based on the molecular weight of HLA-G2, the peak indicated by the arrow was determined as the elution fraction of HLA-G2. The fraction was collected and concentrated, considering the obtained product as HLA-G2.

(Determination of Affinity to Mouse Immunosuppressive Receptor PIR-B)

The affinity of HLA-G2 to Pried-Immunoglobulin-like Receptor B (PIR-B), a mouse homolog of the human immunosuppressive receptor Leukocyte Immunoglobulin-Like Receptor B (LILRB) was evaluated by surface plasmon resonance.

(1) Preparation of PIR-B

HEK293T cells were transfected with the extracellular domain of PIR-B and cultured in DMEM containing 1% FBS for 72 hours. The amino acid sequence of the extracellular domain of PIR-B and the nucleic acid sequence of the gene encoding the domain are shown in SEQ ID NOS: 5 and 6, respectively. The amino acid sequence of the full-length PIR-B is publicly available in NCBI as NM_011095, in which the extracellular domain of PIR-B corresponds to Domains 1 to 6.

Next, the supernatant was collected from the culture and subjected to nickel affinity chromatography to obtain purified extracellular domain of PIR-B. The resulting product was concentrated, and then purified by gel filtration chromatography under the following conditions.

### <Conditions of Gel Filtration Chromatography>

Column: HiLoad 26/60, Superdex 75 (60 cm, id 26 mm)

Mobile phase: 20 mM Tris-HCl, 100 mM NaCl buffer (pH8)

Flow rate: 2.5 ml/min

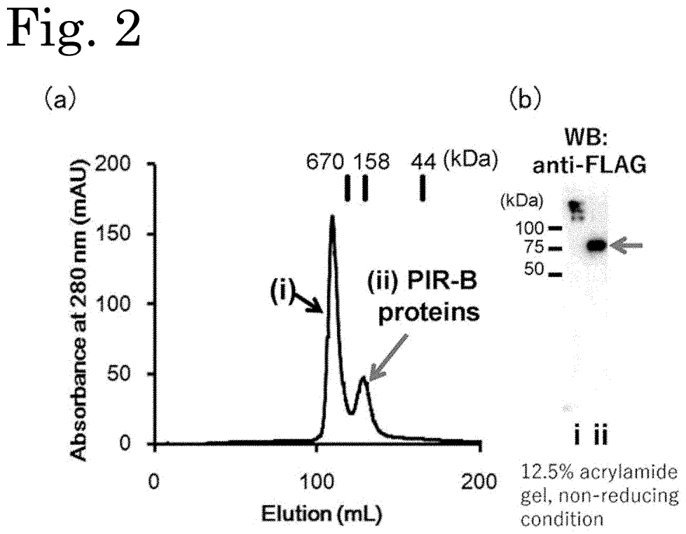

The chromatogram obtained from the gel filtration chromatography is shown in FIG. 2A. As shown in FIG. 2A, two peaks were detected. The peak fractions were taken and subjected to SDS-PAGE under non-reducing conditions (12.5% acrylamide gel). The results are shown in FIG. 2B. Based on the molecular weight of PIR-B, the peak indicated by the arrow was determined as the elution fraction of PIR-B. The fraction was collected and concentrated, considering the obtained product as the extracellular domain of PIR-B.

The purified extracellular domain of PIR-B was dissolved in a Reaction buffer (50 mM D-biotin, 100 mM ATP, 15 μM BirA) so that the concentration was 15 μM, thereby biotinylating the domain. Gel filtration chromatography (Superdex 200) was performed to isolate and purify the biotinylated extracellular domain of PIR-B from the Reaction buffer.

(2) Determination of Affinity of HLA-G2 to PIR-B (Extracellular Domain)

Using BIAcore® 3000 (GE healthcare BIAcore), the affinity between HLA-G2 and the extracellular domain of PIR-B prepared as described above was determined in a surface plasmon resonance experiment. Streptavidin was first covalently fixed on a research grade sensor chip, and then the biotinylated extracellular domain of PIR-B or BSA as a negative control was fixed via the streptavidin. Then, a solution of HLA-G [α1-3] dimer in HBS-EP running buffer (10 mM HEPES pH7.5, 150 mM sodium chloride, 3.4 mM EDTA, 0.005% Surfactant P20) was run at 5 μL/min. Kinetics analysis of binding response was performed at various concentrations by subtracting the response measured in a control flow cell from the response measured in a sample flow cell. BIAevaluation version 4.1.1 (GE Healthcare) was used in the analysis.

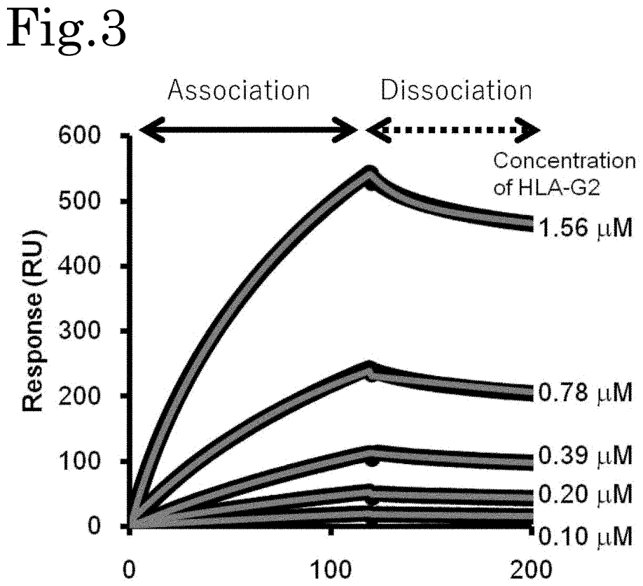

FIG. 3 shows the reaction of PIR-B against various concentrations of HLA-G2 (0.10 μM, 0.20 μM, 0.39 μM, 0.78 μM, and 1.56 μM). As seen from the results in FIG. 3, the analysis using a 1:1 binding model revealed that the apparent dissociation constant (Kd value) was 142 nM. This result demonstrated that HLA-G2 can bind to the extracellular domain of mouse PIR-B that corresponds to human LILRB2.

### Example 2

To demonstrate the function of HLA-G2 protein on human peripheral blood-derived cells, monocytes expressing a receptor LILRB2 were prepared by CD14 positive selection from human PBMCs.

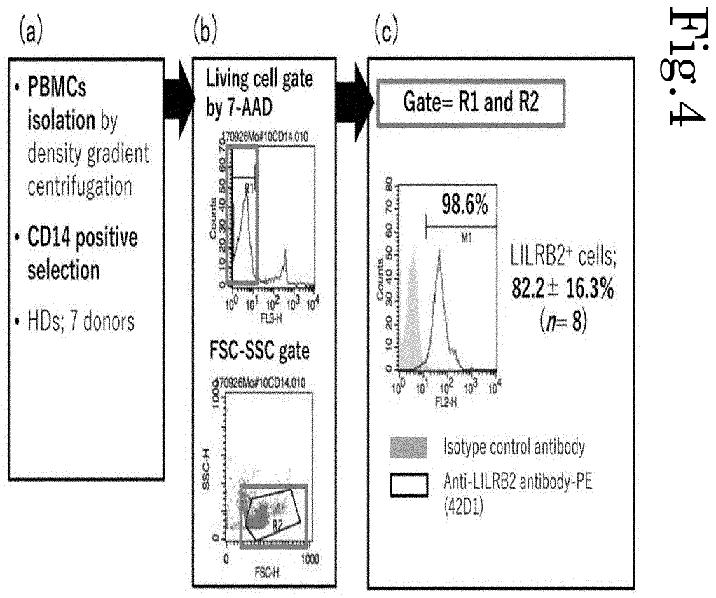

FIG. 4 shows preparation of monocytes expressing LILRB2. Peripheral blood mononuclear cells (PBMC) were isolated by density gradient centrifugation (DGC). The cells were then subjected to CD14 positive selection.

The method of preparing human monocytes expressing LILRB2 will be described. Peripheral blood was collected and diluted with PBS. The diluted peripheral blood was layered on Lymphoprep (solution composed of Axis-Shield, diatrizoate sodium, polysaccharide) and centrifuged. A PBMC layer formed between plasma and Lymphoprep layers was collected. The collected PBMCs was subjected to magnetic cell sorting (MACS) (Miltenyi Biotec) using human CD14 MicroBeads (Miltenyi Biotec), thereby collecting CD14-positive PBMCs as monocytes. FIG. 4C shows the collected monocyte expressed LILRB2. The collected cells were stained using a phycoerythrin (PE)-labeled anti-LILRB2 antibody (42D1), and then further treated with 7-AAD 10 minutes before measurement. Analysis was performed using cells that had not been stained by 7-AAD (R1 gate) and had found in the region in the FSC-SSC dot blot in which monocytes were to appear (R2 gate) (FIG. 4B). The M1 area that does not include the histogram (gray) obtained by staining with the same isotype antibody as the anti-LILRB2 antibody (Isotype Control Antibody) comprises 82.2% cells on an average as stained with an anti-LILRB2 antibody, demonstrating that the CD14-positive monocytes expressed LILR2.

The LILRB2-expressing monocytes prepared as described above were incubated for two days with HLA-G2, followed by flow cytometry to measure the expression levels of cell surface molecules, CD86, HLA-DR, and PD-L1.

To the LILRB2-expressing monocytes was added HLA-G2 (2.3 μM) or control PBS, and the cells were cultured in RPMI-1640 containing 10% FBS at 37° C., 5% CO₂. After two days, the supernatant was collected and cell lysates were prepared for flow cytometry, ELISA, and Western blotting.

The cell culturing protocol and cell lysate preparation method will be described. To LILRB2-expressing monocytes was added 2.3 μM HLA-G2 dissolved in PBS or the same amount of PBS as a control, and the cells were cultured in RPMI-1640 supplemented with 10% FBS and Penicillin-Streptomycin-Amphotericin B Suspension (Wako) at 37° C., 5% CO₂. After two days, the cells were collected for flow cytometry. The culture supernatant was used for ELISA. In addition, about 2×10⁶ cells that had been treated in the same manner were collected, and lysed in RIPA buffer (50 mM Tris-HCl pH 8.0, 150 mM NaCl, 2 mM EDTA, 1% NP-40, 0.5% sodium deoxycholate, 0.1% sodium dodecyl sulfate, protease inhibitor cocktail cOmplete (Roche), phosphatase inhibitor (Wako)) to prepare a cell lysate.

The flow cytometry (FCM) protocol will be described. Cells were suspended in FCM buffer (a solution of 0.5% BSA and 0.05% sodium azide in PBS), followed by addition of a PE- or FITC-labeled antibody. The cells were incubated in the dark at room temperature for 15 minutes and washed two times with FCM buffer for the measurement. The cells were treated with 7-AAD ten minutes before the measurement. Gate settings were made as described above, and comparison of mean fluorescence intensities (MFI) of obtained histograms was performed.

The ELISA protocol will be described. R&D Systems ELISA kit DuoSet was used in the measurement according to the instruction manual. A capture antibody was fixed on a 96-well plate overnight at room temperature, and the plate was washed three times with 0.1% Tween-20/PBS solution (PBST). The plate was blocked with 1% BSA/PBS solution for 1 hour, washed three times with PBST, followed by addition of cell supernatants into the wells. After 2 hours, the plate was washed three times with PBST, followed by addition of a Detection antibody and incubation for 2 hours. The plate was washed three times with PBST before addition of an HRP-conjugated streptavidin solution and incubation for 20 minutes, followed by washing three times with PBST. After TMB solution (Thermo Fisher Scientific) was added and incubated for 15 to 25 minutes, 2N sulfuric acid was added to stop the reaction. The absorbance at 450 nm was measured with a plate reader (absorbance at 540 nm was subtracted as reference wavelength). Calibration curves were prepared using standard cytokine reagents to determine the amounts of the cytokines in the cell culture supernatant. The cell culture supernatant was used with dilution as appropriate with 1% BSA/PBS solution.

The western blotting protocol will be described. Cell lysates were electrophoresed in SDS-PAGE using 12.5% or 10% acrylamide gel. The cell lysate contained 5% mercaptoethanol-containing sample buffer (final concentrations: 63 mM Tris-HCl pH6.8, 2% sodium dodecyl sulfate, 10% glycerol, and 0.005% bromophenol blue). Proteins separated in the gel were transferred to a PVDF membrane, which was then blocked with TBS solution containing 5% skim milk. Thereafter, according to the instruction manuals of the antibodies used in Western blotting, the membrane was treated with the antibodies. For detection of bands, chemiluminescence was detected using ECL prime (GE Healthcare), LAS 4000 mini.

Conditions for flow cytometry, ELISA, and Western blotting are shown below.

Flow Cytometry

Antibodies: anti-HLA-DR antibody (Immu-357), anti-CD86 antibody (2331(FUN-1)), and anti-PD-L1 antibody (29E.2A3);

ELISA

Kit: DuoSet ELISA human IL-6 and IL-10 (R&D Systems);

Western Blotting

Antibodies: anti-STAT3 antibody (79D7), anti-phospho-STAT3 (Tyr705) antibody (polyclonal), anti-IDO antibody (D5J4E), and anti-β-actin antibody (8H10D10).

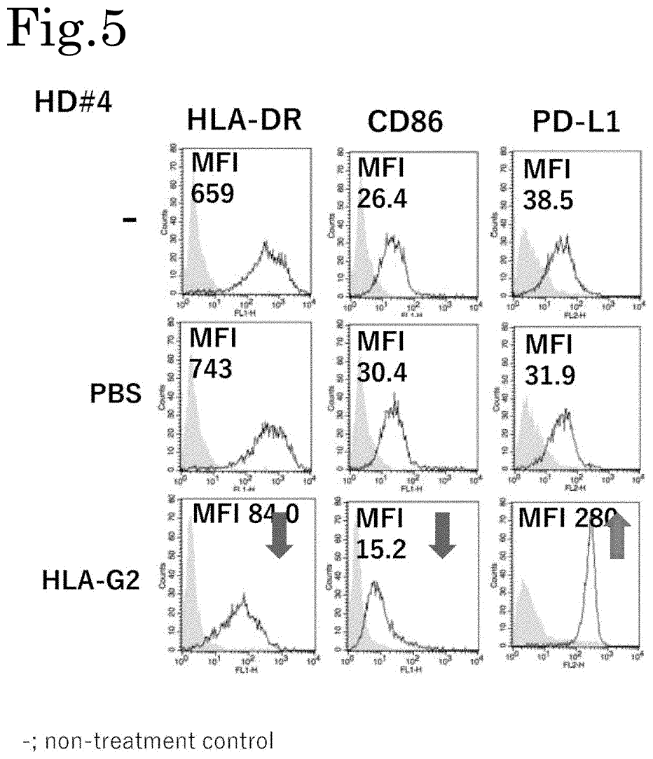

The results of the flow cytometry are shown in FIG. 5. The human LILRB2-expressing monocytes after two days of incubation with HLA-G2 showed down-regulation of CD86 and HLA-DR and significant up-regulation of PD-L1. Thus, the human LILRB2-expressing monocytes after two days of incubation with HLA-G2 exhibited immunosuppressive phenotype.

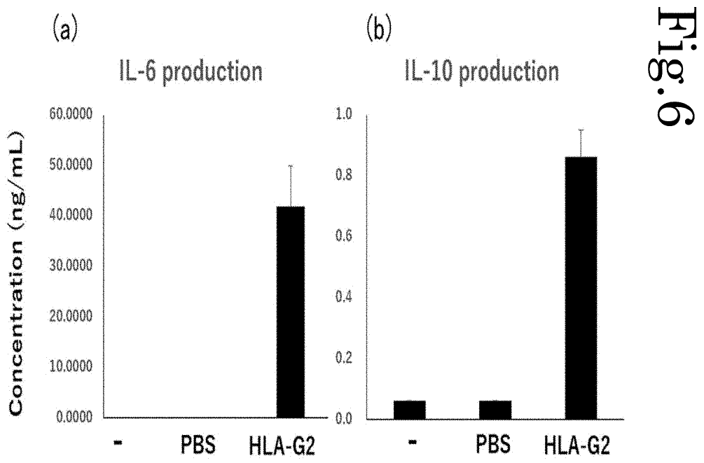

The results of the ELISA are shown in FIG. 6. The human LILRB2-expressing monocytes after two days of incubation with HLA-G2 showed induced production of cytokines IL-10 and IL-6.

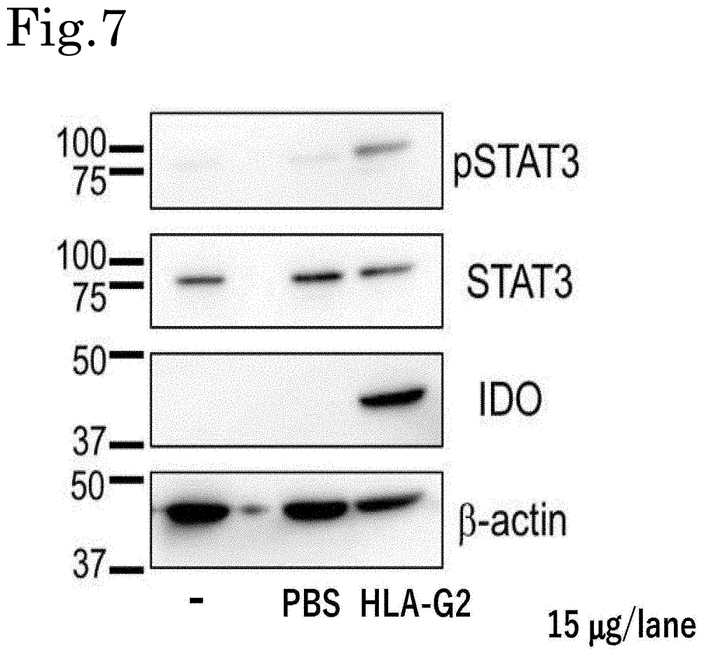

The results of the Western blotting are shown in FIG. 7. The human LILRB2-expressing monocytes after two days of incubation with HLA-G2 showed increased phosphorylation of intracellular protein STAT3 and up-regulation of IDO.

### Example 3

Next, we investigated what happened when the interaction between LILRB2 and HLA-G2 was blocked. As an anti-LILRB2 antibody that blocks the interaction between LILRB2 and HLA-G1, use of 27D6 (CD85d (ILT4) monoclonal antibody (27D6), functional grade, eBioscience (Thermo Fisher Scientific)) was tested. 27D6 is an anti-LILRB2 antibody that blocks binding of HLA-G1 to LILRB2 (Allan D S et al., Tetrameric complexes of human histocompatibility leukocyte antigen (HLA)-G bind to peripheral blood myelomonocytic cells. J Exp Med.1999 Apr. 5; 189 (7): 1149-56). First, we investigated whether or not 27D6 blocks the interaction between LILRB2 and HLA-G2.

The SPR analysis protocol will be described. Measurement was performed using CAP chip and BIACORE 2000 (GE Healthcare) at 25° C. HBS-EP buffer was used as a running buffer. First, each about 700 RU of biotinylated LILRB2 was immobilized on two flow cell chips. Addition of 10 nM 27D6 antibody to only one of the flow cells was repeated three times until the binding reaches saturation. Then, 3.6 μM HLA-G2 was injected into the two LILRB2-immobilized flow cells, and the responses were compared (a response of a BSA-immobilized flow cell was used as a control).

Anti-LILRB2 antibody (27D6): 10 nM

HLA-G2: 3.6 μM

HBS-EP buffer: 10 mM HEPES, 150 mM NaCl, 3 mM EDTA, 0.005% Tween-20

Measuring apparatus: BIACORE3000 (GE)

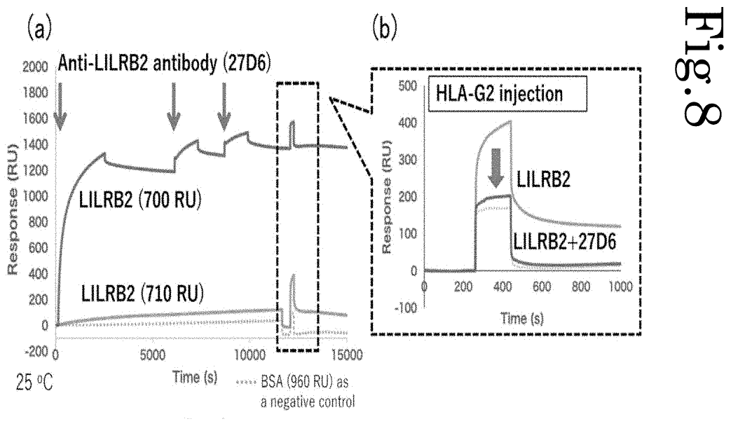

The results are shown in FIG. 8. It was found that injection of HLA-G2 resulted in reduced response for LILRB2+27D6 as compared with LILRB2 alone (FIG. 8B). Thus, it was demonstrated that 27D6 blocked the interaction between LILRB2 and HLA-G2.

Next, analysis of blocking of HLA-G2-treated monocytes was performed. Cells were incubated with 4 g of 27D6 for 30 minutes followed by two days of incubation with HLA-G2. More specifically, after 30 minutes of incubation with 4 g of 27D6 antibody (the same amount of an isotype-matched antibody was used as a control), 2.3 μM HLA-G2 dissolved in PBS or the same amount of PBS as a control was added, and the cells were cultured in RPMI-1640 supplemented with 10% FBS and Penicillin-Streptomycin-Amphotericin B Suspension (Wako) at 37° C., 5% CO₂ for two days. The cells were collected and lysed in RIPA buffer. Subsequent operations, such as Western blotting, were performed in the same manner as described above.

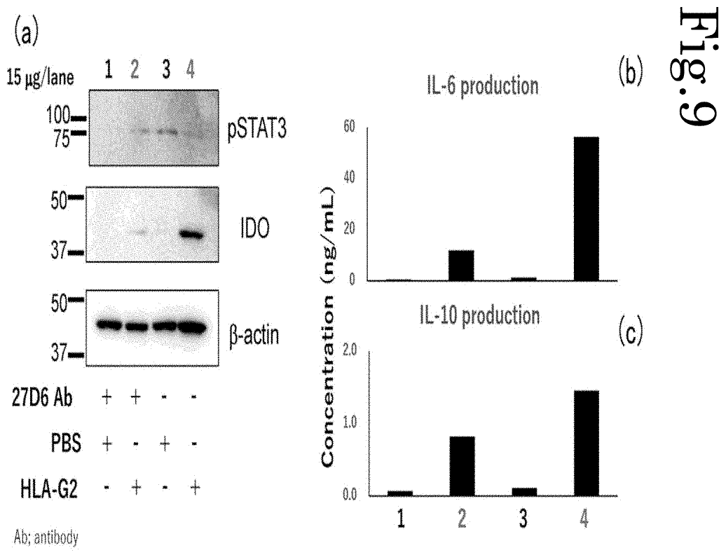

The results are shown in FIG. 9. The incubation with 27D6 resulted in down-regulation of IDO (FIG. 9A). In addition, the incubation with 27D6 resulted in reduced production of IL-10 and IL-6 (FIGS. 9B and 9C). Thus, it was suggested that 27D6 blocks the interaction between LILRB2 and HLA-G2, thereby reducing the expression of IDO and the production of IL-10 and IL-6.

### Example 4

Next, using IL-4-DC, dendritic cells derived from human monocytes, we studied the events caused by binging of HLA-G2 to LILRB2.

IL-4-DC was incubated in a medium supplemented with 1,000 U/mL GM-CSF and 500 U/mL IL-4 for six days. The IL-4-DC was cultured with HLA-G2 (2.3 μL) or PBS control in a medium (10% FBS RPMI-1640 (supplemented with antibiotics)+1,000 U/mL GM-CSF and 500 U/mL IL-4) at 37° C., 5% CO₂. Three days after the start of culture, the medium was replaced, and the supernatant was collected for ELISA. Six days after the start of culture, the supernatant was collected for flow cytometry.

Conditions for flow cytometry, ELISA, and Western blotting are shown below.

Flow Cytometry

Antibodies: anti-HLA-DR antibody (Immu-357), anti-CD86 antibody (2331(FUN-1)), and anti-PD-L1 antibody (29E.2A3)

ELISA

Kit: DuoSet ELISA human IL-6 and IL-10 (R&D Systems)

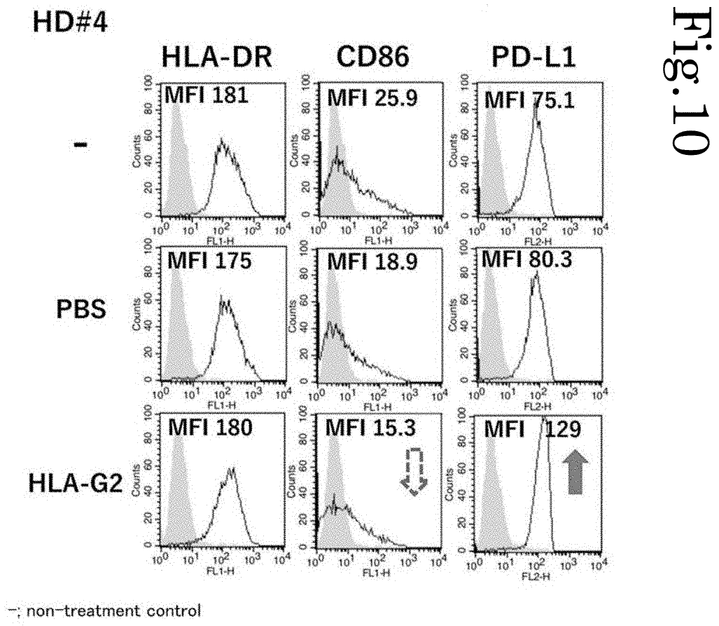

The results of the flow cytometry are shown in FIG. 10. The IL-4-DC after two days of incubation with HLA-G2 showed down-regulation of CD86 and significant up-regulation of PD-L1. Thus, IL-4-DC after six days of incubation with HLA-G2 also exhibited immunosuppressive phenotype.

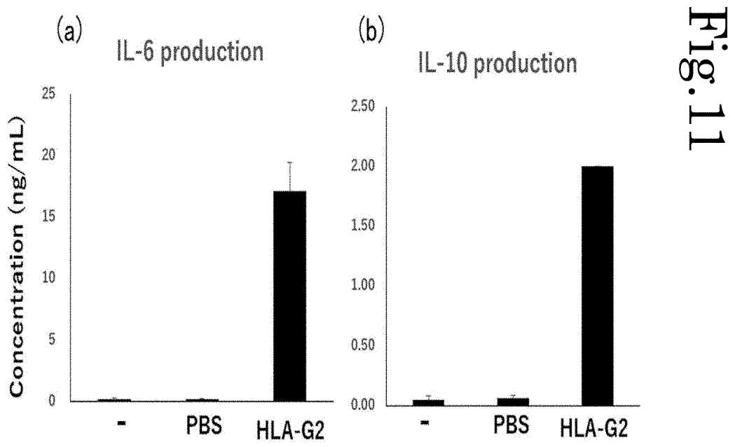

The results of the ELISA are shown in FIG. 11. The IL-4-DC after three days of incubation with HLA-G2 also showed induced production of cytokines IL-10 and IL-6.

### Example 5

Next, using IFN-DC, dendritic cells derived from human monocytes, we studied the events caused by binging of HLA-G2 to LILRB2.

IFN-DC was cultured according to Nieda M et al., Exp Dermatol. 2015 January; 24 (1): 35-41. doi: 10.1111/exd.12581. Epub 2014 Dec. 8. Two days after the start of culture, the cells were cultured with HLA-G2 (2.3 μM) or PBS control in a medium (10% FBS RPMI-1640 (supplemented with antibiotics)+1,000 U/mL GM-CSF and IFN-α) at 37° C., 5% CO₂. Four days after the start of culture, the medium was replaced, and the supernatant was collected for flow cytometry and ELISA.

Conditions for flow cytometry, ELISA, and Western blotting are shown below.

Flow Cytometry

Antibodies: anti-HLA-DR antibody (Immu-357), anti-CD86 antibody (2331(FUN-1)), and anti-PD-L1 antibody (29E.2A3)

ELISA

Kit: OptEIA ELISA human IL-6 and IL-10 (BD)

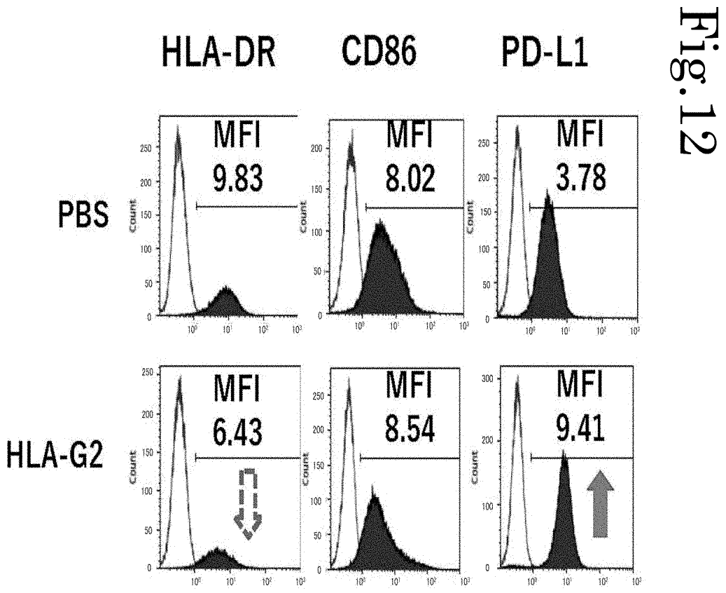

The results of the flow cytometry are shown in FIG. 12. The IFN-DC after two days of incubation with HLA-G2 showed down-regulation of HLA-DR and significant up-regulation of PD-L1. Thus, IFN-DC after two days of incubation with HLA-G2 also exhibited immunosuppressive phenotype.

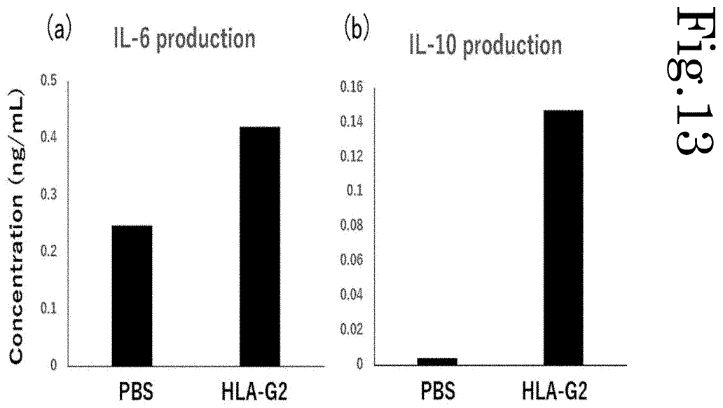

The results of the ELISA are shown in FIG. 13. The IFN-DC after two days of incubation with HLA-G2 also showed induced production of cytokines IL-10 and IL-6.

### Example 6

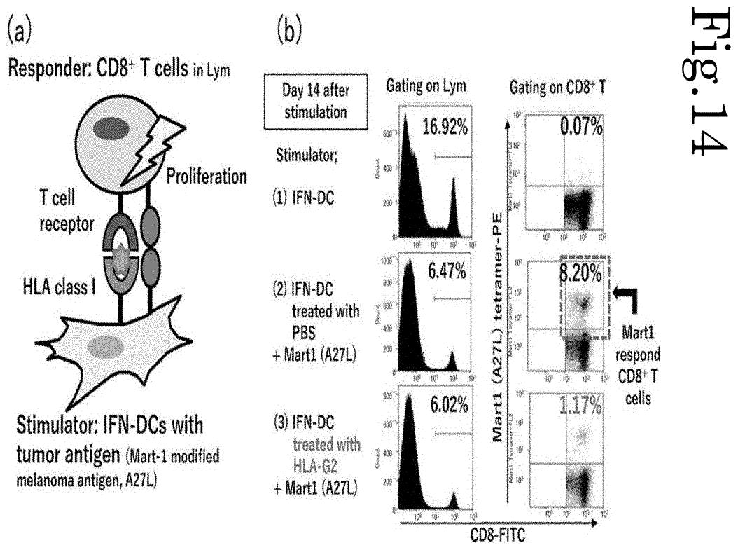

Next, IFN-DC was subjected to an autologous mixed lymphocyte reaction experiment using CD8⁺ T cells (FIG. 14A).

IFN-DC after two days of incubation with HLA-G2 or PBS (control) was treated with Mart1 (A27L) peptide overnight. Mart1 (A27L) peptide (ELAGIGILTV: SEQ ID NO: 7) is a peptide derived from a melanoma-related antigen Melan-A/MART1, and presented on HLA-A*0201. The peptide is modified to be more easily recognized by CD8⁺ T cells by substituting alanine as the 27th amino acid with leucine. IFN-DC and nonadherent PBMC (used as lymphocytes) were mixed at 1:10, and cultured in a 10% FBS RPMI-1640 medium in the presence of 100 U/mL IL-2 for 14 days with repeated passage every 3 days. Thereafter, the cells were stained using FITC-labeled anti-CD8 antibody and PE-labeled Mart1 (A27L) peptide tetramer for flow cytometry analysis. The flow cytometry was performed by double staining with HLA-A2/Mart1 (A27L) tetramer (using PE-conjugated SA) and anti-CD8 (B9.11)-FITC (Beckman Coulter) using Epics XL MCL (Beckman Coulter, Brea, Calif., USA).

The results are shown in FIG. 14B. CD8⁺ T cells were activated in the absence of HLA-G2. However, reduction of the activation of CD8⁺ T cells was observed by treatment with HLA-G2. Thus, it was suggested that HLA-G2 induced suppression of tumor immunity and blocking of the interaction between HLA-G2 and LILRB2 activated tumor immunity.

### Example 7

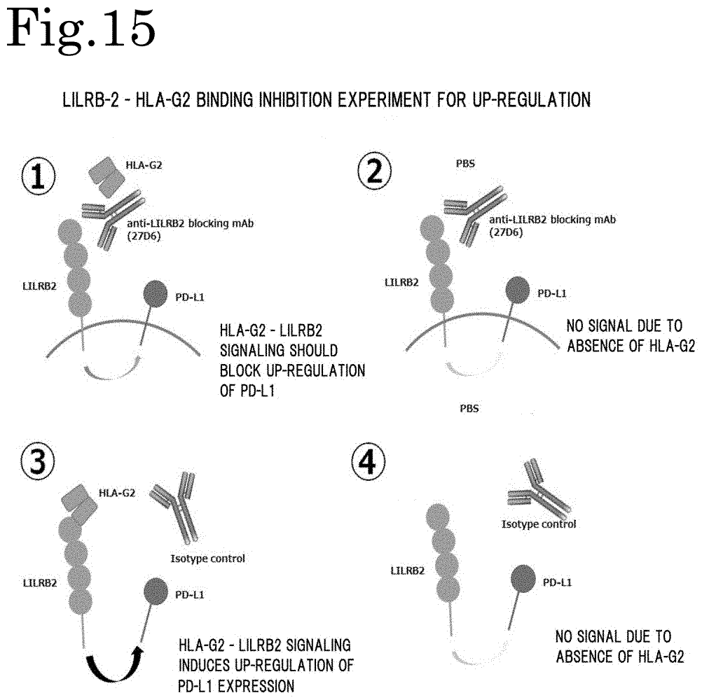

Next, up-regulation of PD-L1 was studied by LILRB2-HLA-G2 binding inhibition experiment. The outline of the experiment is shown in FIG. 15. LILRB2-expressing human monocytes derived from Healthy Controls 1 and 2 (Donors 1 and 2) were incubated with 27D6 (or control antibody) for 30 minutes. 27D6 was used in an amount of 10 g for Healthy Control 1, while in an amount of 7 g for Healthy Control 2. The same amount of an isotype-matched antibody was used as the control. The method of preparing LILRB2-expressing human monocytes from Healthy Controls 1 and 2 was in the same manner as in Example 2. After the preparation, 2.3 μM HLA-G2 dissolved in PBS or the same amount of PBS as a control was added, and the cells were cultured in RPMI-1640 supplemented with 10% FBS and Penicillin-Streptomycin-Amphotericin B Suspension (Wako) at 37° C., 5% CO₂ for two days. Thereafter, the cells were stained with PE-labeled anti-PD-L1 antibody for flow cytometry analysis. The flow cytometry was performed using anti-PD-L1 antibody (29E.2A3). The mean fluorescence intensity and the percentage of PD-L1⁺ cells (%) of the histogram of the cells stained with PE-labeled anti-PD-L1 antibody were compared to determine the percent increase in PD-L1 expression in the presence and absence of 27D6 antibody.

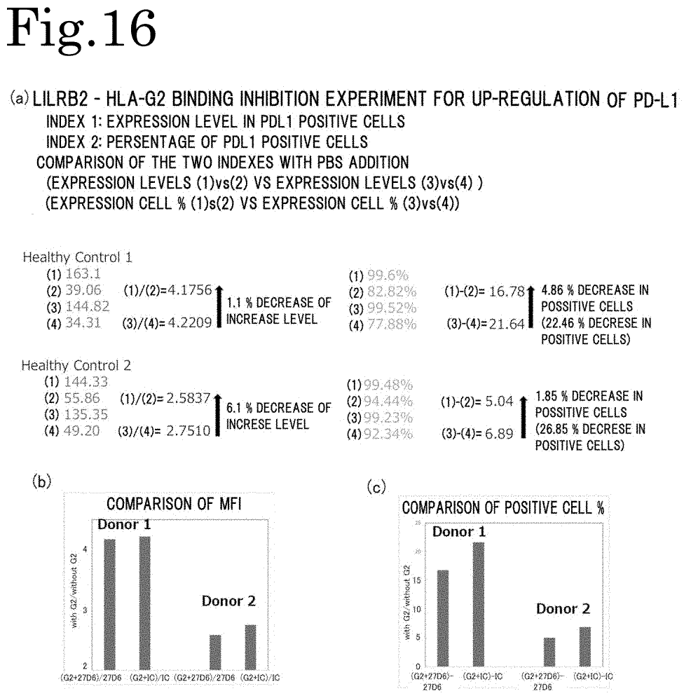

The results are shown in FIGS. 16A to 16C. FIG. 16B shows Mean Fluorescence Intensity (MFI: difference in mean fluorescence intensity from isotype control) for Donor 1 (Healthy Control 1) and Donor 2 (Healthy Control 2), and FIG. 16C shows the percent decrease in PLD1 positive cells for Donor 1 (Healthy Control 1) and Donor 2 (Healthy Control 2). In FIGS. 16B and 16C, “G2” represents “HLA-G2”, and “IC” represents “isotype control.” It was demonstrated that, in both Donor 1 (Healthy Control 1) and Donor 2 (Healthy Control 2), 27D6 blocked the interaction between HLA-G2 and LILRB2 and reduced the expression of PD-L1.

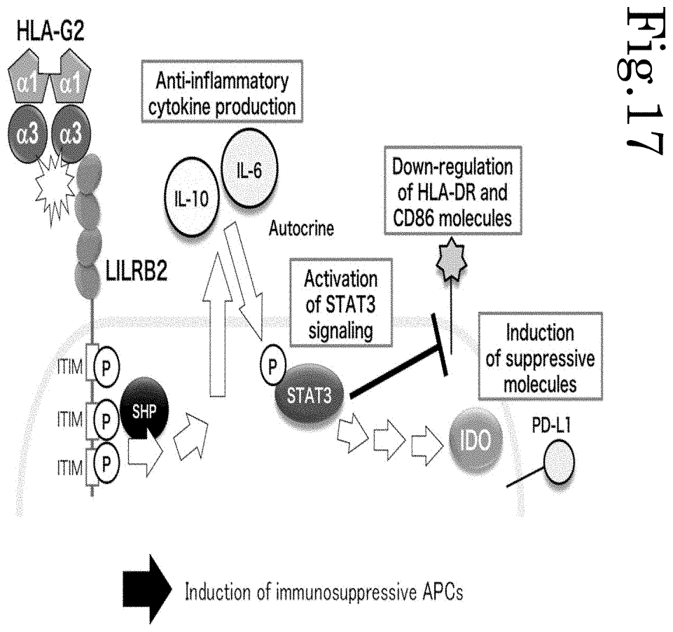

FIG. 17 illustrates a tumor immunity mechanism induced by blocking the interaction between HLA-G2 and LLRB2. Blocking the interaction between HLA-G2 and LLRB2 induces tumor immunity via down-regulation of IDO involved in inhibition of T-cell activation, down-regulation of IL-10 that is thought to be an upstream signal of IDO, down-regulation of IL-6 involved in induction of immunosuppression in monocytes and antigen-presenting cells, and down-regulation of PD-L1. Thus, blocking the interaction between HLA-G2 and LLRB2 shows tumor immunity effects through a comprehensive action by, for example, down-regulation of PD-L1 as well as down-regulation of IL-10 and IL-6.

In the future, it is expected to develop screening methods for small-molecule compounds, antibodies, and the like that cancel suppression of tumor immunity induced by HLA-G2-LILRB2 signaling and to ultimately develop novel anticancer drugs targeting HLA-G2-LILRB2 signaling.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

This application claims the benefit of Japanese Patent Application No. 2018-046024, filed on Mar. 13, 2018, the entire disclosure of which is incorporated by reference herein.

1. A prophylactic or therapeutic agent for tumors comprising an inhibitor of an interaction between HLA-G2 and leukocyte Ig-like receptor B2 (LILRB2) as an active ingredient.

2. The prophylactic or therapeutic agent for tumors according to claim 1, wherein the interaction inhibitor is an anti-LILRB2 antibody.

3. A programmed cell death ligand 1 (PD-L1) inhibitor comprising an inhibitor of interaction between HLA-G2 and leukocyte Ig-like receptor B2 (LILRB2) as an active ingredient.

4. The PD-L1 inhibitor according to claim 3, wherein the interaction inhibitor is an anti-LILRB2 antibody.

5. A method of screening for prophylactic or therapeutic agents for tumors, comprising the steps of:
determining a degree of binding between HLA-G2 and leukocyte Ig-like receptor B2 (LILRB2) in the presence and absence of a test substance;
comparing the degree in the presence of the test substance with the degree in the absence of the test substance; and
identifying the test substance as a prophylactic or therapeutic agent for tumors when the degree in the presence of the test substance is lower than the degree in the absence of the test substance.

6. A method of screening for programmed cell death ligand 1 (PD-L1) inhibitors, comprising the steps of:
determining a degree of binding between HLA-G2 and leukocyte Ig-like receptor B2 (LILRB2) in the presence and absence of a test substance;
comparing the degree in the presence of the test substance with the degree in the absence of the test substance; and
identifying the test substance as a prophylactic or therapeutic agent for tumors when the degree in the presence of the test substance is lower than the degree in the absence of the test substance.
